(19) United States
(12) Patent Application Publication
(10) Pub. No.: US 2018/0251815 A1
(43) Pub. Date: Sep. 6, 2018

(54) OXIDIZING AGENT FOR 5-HYDROXYMETHYLCYTOSINE AND METHOD FOR ANALYZING 5-HYDROXYMETHYLCYTOSINE

(71) Applicant: THE UNIVERSITY OF TOKYO, Tokyo (JP)

(72) Inventors: Akimitsu OKAMOTO, Tokyo (JP); Taku MATSUSHITA, Tokyo (JP)

(73) Assignee: The University of Tokyo, Tokyo (JP)

(21) Appl. No.: 15/756,843

(22) PCT Filed: Sep. 2, 2016
(86) PCT No.: PCT/JP2016/075897

§ 371 Date: Mar. 1, 2018

(30) Foreign Application Priority Data
Sep. 4, 2015 (JP) 2015-175121

(51) Int. Cl. C12Q 1/6806; C12Q 1/6869

(52) U.S. Cl. CPC C12Q 1/6806; C12Q 1/6869

(57) ABSTRACT

The purpose of the present invention is to develop and provide a less expensive and novel oxidizing agent for 5-hydroxymethylcytosine, which can selectively oxidize 5-hydroxymethylcytosine on DNA into 5-formylcytosine while preventing the DNA structure from destabilization and suppressing side reactions, and a method for detecting a demethylation site at a high accuracy in demethylation of DNA with the use of the aforesaid oxidizing agent.

Provided is an oxidizing agent for 5-hydroxymethylcytosine that comprises a nitroxyl radical molecule and a copper salt or a copper complex, and/or a nitroxyl radical molecule-copper complex.

### TECHNICAL FIELD

The present invention relates to: an agent for oxidizing 5-hydroxymethylcytosine (which is often referred to as “5hmC” in the present description) that is generated upon demethylation of DNA or the like; a DNA demethylation analysis reagent for detecting 5-formylcytosine (which is often referred to as “5fC” in the present description) that is generated by the oxidation of 5hmC and identifying a DNA demethylation site; and a method of analyzing 5hmC serving as an indicator of demethylation of DNA, using the detection reagent.

### BACKGROUND ART

Methylation of DNA, which is a form of DNA modification, is carried out by methylating the carbon at position 5 of cytosine by a DNA methyltransferase (DNMT) in a CpG sequence consisting of a phosphodiester bond between cytosine and guanine on DNA, and converting it to 5-methylcytosine (which is often referred to as “5mC” in the present description) (FIG. 1). The methyl group in 5mC is not involved in formation of a hydrogen bond between a base pair, but it affects the interaction between a protein and DNA. Thus, in methylated DNA, gene expression may be inactivated. Accordingly, in organisms, gene expression and the differentiation and aging of cells or tissues are epigenetically regulated by methylation of DNA (Non Patent Literatures 1 to 6).

On the other hand, demethylation of DNA is completed by removing a methyl group from 5mC by the catalytic activity of an enzyme, such as TET (Ten-Eleven-Translocation), and converting 5mC to the original cytosine (FIG. 1). It has been clarified that, in a demethylation reaction in vivo, the methyl group is not removed at once, but it is converted to cytosine via multi-step hydroxylation reactions from 5mC to 5hmC, then to 5fC, and then to 5-carboxylcytosine (5caC) (Non Patent Literatures 7 to 10). However, with regard to the specific mechanism of each reaction, there are still many unknown points. DNA demethylation is a change on DNA, which is of great importance for reactivation of gene function including the reprogramming of cells such as germ cells.

If epigenetic control mechanism by DNA methylation and DNA demethylation could be elucidated, it would become possible to monitor and profile gene expression or the differentiation or aging of cells, and such a technique could also be applied to the fields of regenerative medicine using iPS cells or ES cells, cancer therapy, basic research of epigenetics, etc. Therefore, it has been desired to develop a method of efficiently analyzing DNA methylation or DNA demethylation.

In the case of analyzing DNA methylation, relatively inexpensive analysis methods of high accuracy, such as a bisulfite sequencing method, a qAMP (quantitative analysis of DNA methylation using real-time PCR) method, a COBRA (combined bisulfite restriction analysis) method, and a methylated DNA immunoprecipitation method, have been developed. On the other hand, in the case of DNA demethylation, such an analysis method has not yet been developed. In DNA demethylation, detection of 5hmC that is an intermediate product generated at an initial step of multi-step hydroxylation reactions is a key for the analysis of the DNA demethylation, and thus, it is important to develop a method of specifically detecting this substance at a low cost.

To date, as methods of detecting 5hmC, a method of modifying the hydroxy group of 5hmC with sugar (Non Patent Literatures 11 to 13), a method of capturing 5hmC with an anti-5hmC antibody (Non Patent Literature 14), and a method of using a ruthenate (Non Patent Literature 15) have been known.

However, the sugar modification method has been problematic in that the results are easily influenced by the activity of enzyme that catalyzes sugar modification, or in that analysis accuracy is low.

Moreover, the antibody capturing method can recover a DNA fragment containing 5hmC, but it cannot identify the position of 5hmC in the DNA fragment. As such, this method has been problematic in that the operation to identify the position of 5hmC must be carried out, separately, after the recovery of the DNA fragment.

The method of using a ruthenate is used most commonly, at present, as a method of detecting 5hmC. This method is based on the principle that the hydroxy group of 5hmC is oxidized by ruthenate such as potassium perruthenate, and that the generated 5fC is then detected. However, since ruthenium is a rare metal, ruthenate is expensive, and thus, the method of using a ruthenate has been problematic in terms of high costs. Moreover, this method has also been problematic in that since ruthenate non-specifically oxidizes hydroxy groups, there are many side reactions to other hydroxy groups in a biomolecule. Furthermore, side effects such as destabilization or degradation of a DNA structure have also been suggested.

As mentioned above, all of the conventional 5hmC detection methods have had insufficient detection accuracy, and thus, have caused a high false-positive rate. If a demethylation site on DNA could be specifically detected, it would become easy to induce differentiation by a gene-specific manipulation, or to design a gene targeted therapy for disease. Accordingly, it has been desired to develop an inexpensive DNA demethylation detection method of higher accuracy.

### CITATION LIST

### Non Patent Literature

Non Patent Literature 1: Jones P. L. and Wolffe A. P., 1999, Semin. Cancer Biol., 9: 339-347.
Non Patent Literature 2: Tate P. H. and Bird A. P., 1993, Curr. Opin, Genet. Dev., 3: 226-231.
Non Patent Literature 3: Colot V. and Rossignol J. L, 1999, BioEssays, 21: 402-411.
Non Patent Literature 4: Feil R. and Khosla S., 1999, Trend. Genet., 15: 431-435.
Non Patent Literature 5: Jones P. A. and Laird P. W., Nature Genet., 1999, 21: 163-167.
Non Patent Literature 6: Robertson K. D., 2005, Nat. Rev. Genet., 6: 597-610.
Non Patent Literature 7: Kriaucionis S. and Heintz N., 2009, Science: 324, 929.
Non Patent Literature 8: Tahiliani M. et al., 2009, Science: 324, 930.
Non Patent Literature 9: Globisch D. et al., 2010, PLoS ONE, 5, e15367.
Non Patent Literature 10: Wu S. C. and Zhang Y., 2010, Nat. Rev. Mol. Cell Biol., 11: 607.
Non Patent Literature 11: Song C.-X. et al., 2011, Nature Biotech., 29: 68.
Non Patent Literature 12: Szwagierczak A. et al., 2010, Nucleic Acids Res., 38, e181
Non Patent Literature 13: Liutkeviciute Z. et al., 2011, Angew. Chem, Int. Ed., 50: 2090.
Non Patent Literature 14: Ito S. et al., 2010, Nature, 466: 1129.
Non Patent Literature 15: Booth M. J., et al., 2012, Science 336: 934-937.

### SUMMARY OF INVENTION

### Technical Problem

It is an object of the present invention to develop and provide a less expensive and novel oxidizing agent for 5-hmC, which can selectively oxidize 5hmC on DNA into 5fC while preventing side reactions.

It is another object of the present invention to develop and provide a DNA demethylation analysis method capable of reducing a false-positive rate in the detection of DNA demethylation, and detecting a demethylation site with high accuracy.

### Solution to Problem

5hmC includes an allyl alcohol structure (in the broken line frame in FIG. 1). In enormous types of biomolecules, a molecule having an allyl alcohol structure is very rare.

Hence, the present inventors have thought that such an allyl alcohol structure could become a target site of a 5hmC-specific reaction, and have searched for an easily available, inexpensive reagent capable of selectively oxidizing a hydroxy group in the structure. As a result, the present inventors have developed a novel method of using a nitroxyl radical molecule and a copper ion as an oxidizing agent for 5hmC, and thereby selectively oxidizing a hydroxy group in the allyl alcohol structure of 5hmC, so as to convert 5hmC to 5fC. The present invention is based on the development results, and provides the following.

(1) An oxidizing agent for 5hmC, consisting of the following (a) and/or (b):

(a) a nitroxyl radical molecule and a copper salt or a copper complex; and/or

(b) a nitroxyl radical molecule-copper complex.

(2) The oxidizing agent for 5hmC according to the above (1), further comprising the following (c):

(c) one or more reaction promoters selected from the group consisting of pyridine, bipyridine, phenanthroline, ethylenediamine, propanediamine, imidazole, and a derivative thereof.

(3) The oxidizing agent for 5hmC according to the above (1), wherein the nitroxyl radical molecule is 2,2,6,6-tetramethylpiperidine-1-oxyl, 3-carbamoyl-2,2,5,5-tetramethyl-3-pyrrolin-1-yloxy, 2-azaadamantane-N-oxyl, 9-azabicyclo[3.3.1]nonane-N′-oxyl, or a derivative thereof.
(4) A DNA demethylation analysis reagent, comprising the oxidizing agent for 5hmC according to any one of the above (1) to (3).
(5) The DNA demethylation analysis reagent according to the above (4), further comprising a bisulfite.
(6) A DNA demethylation analysis kit, comprising the DNA demethylation analysis reagent according to the above (5).
(7) A method of oxidizing 5hmC, comprising a mixing step of mixing a test substance that may contain 5hmC with the oxidizing agent for 5hmC according to any one of the above (1) to (3) in a reaction solution, and an oxidation step of incubating the reaction solution at a temperature of 4° C. to 90° C. for 1 to 100 hours to oxidize a hydroxy group in an allyl alcohol structure contained in 5hmC.
(8) A method of analyzing 5hmC, comprising a mixing step of mixing DNA with the oxidizing agent for 5hmC according to any one of the above (1) to (3) in a reaction solution, an oxidation step of incubating the reaction solution at a temperature of 4° C. to 90° C. for 1 to 100 hours to oxidize a hydroxy group in an allyl alcohol structure constituting 5hmC, and a detection step of detecting 5 fC generated in the oxidation step.
(9) The method according to the above (8), comprising a hydrogen bond cleavage step of cleaving a hydrogen bond contained in DNA, before performing the mixing step.
(10) The method according to the above (9), comprising a DNA extraction step of extracting DNA from a biological sample, before performing the hydrogen bond cleavage step.
(11) The method according to the above (9) or (10), wherein, in the hydrogen bond cleavage step, DNA is dissolved in a basic solution to cleave a hydrogen bond.
(12) The method according to any one of the above (8) to (11), wherein, in the detection step, 5fC is detected by a bisulfite sequencing method.

The present description incorporates the contents disclosed in Japanese Patent Application No. 2015-175121, to which the present application claims priority.

### Advantageous Effects of Invention

The oxidizing agent for 5hmC of the present invention is inexpensive, and also, can selectively oxidize only a hydroxy group in an allyl alcohol structure present in 5hmC and can convert it to 5fC.

According to the DNA demethylation analysis reagent of the present invention and the method of analyzing 5hmC using the aforementioned reagent, 5hmC generated by DNA demethylation can be converted to 5fC without affecting the DNA structure, and 5fC can be then detected. Moreover, based on the results, a DNA demethylation site can be detected with high accuracy.

### BRIEF DESCRIPTION OF DRAWINGS

FIG. 1 is a conceptual diagram showing the methylation process and demethylation process of a cytosine residue in a living body. The broken line frame in the structural formula of 5hmC indicates an allyl alcohol structure.

FIG. 2 is a conceptual diagram showing reactions that may occur in Example 1. X in the nucleotide sequence of target DNA represents 5hmC, and Y represents 5fC. If 5fC is not generated by the oxidizing agent for 5hmC of the present invention, a cleavage reaction by piperidine does not take place, and the target DNA remains as is, as shown with the arrow in the bottom.

FIG. 3 shows an image obtained with fluorescein for the gel after the electrophoresis in Example 1. This figure shows the results of a cleavage reaction by a piperidine treatment. DNA (15 mer) indicates the 15 mer DNA shown in FIG. 2, which has been added as target DNA upon initiation of the reaction, and DNA (7 mer) indicates 7 mer DNA on the 5′-terminal side, which is predicted to be generated through the cleavage reaction by a piperidine treatment (corresponding to cleaved DNA 1 in FIG. 2).

FIG. 4 is a diagram showing the fluorescence intensity of the bands of 15 mer (A) and 7 mer (B) in the electrophoresis image of FIG. 3 as a graph.

FIG. 5A shows the results obtained by treating (a) deoxy-5-hydroxymethylcytidine (d5hmC) and (b) deoxy-5-methylcytidine (d5mC) with the oxidizing agent for 5hmC of the present invention, and then analyzing over time by HPLC. In the figure, d5fC indicates deoxy-5-formylcytidine.

FIG. 5B shows the results obtained by treating deoxycytidine (dC), deoxythymidine (dT), deoxyguanosine (dG), and deoxyadenosine (dA) with the oxidizing agent for 5hmC of the present invention, and then analyzing over time by HPLC.

FIG. 6A shows the results of the analysis by quantitative PCR. FIG. 6B is a magnified view of the 21-26 cycle region of PCR (the inside of the frame in FIG. 6A) in FIG. 6A. The solid line indicates the results obtained by analyzing untreated DNA as a control (Control), the broken line indicates the results obtained by analyzing DNA treated with the oxidizing agent for 5hmC of the present invention, and the dotted line indicates the results obtained by analyzing DNA treated with potassium perruthenate.

FIG. 7 is a conceptual diagram showing the flow of the double experiment which was performed in Example 4 for individual 5hmC detection methods.

FIG. 8 is a conceptual diagram showing the detection principles of individual methods of detecting a methylation site. Detection principles are shown for a bisulfite sequencing method in FIG. 8A, a 5hmc oxidation method (including the method of the present invention and a ruthenium method) in FIG. 8B, and a TAB-seq method in FIG. 8C.

FIG. 9 shows the reproducibility of individual 5hmC detection methods performed in Example 4. Results of the reproducibility are shown for a bisulfite sequencing method in FIG. 9A, the method of oxidizing 5hmC of the present invention in FIG. 9B, a ruthenium method in FIG. 9C, and a TAB-seq method in FIG. 9D.

FIG. 10 shows the results of Example 5. FIG. 10A shows the results of the method of oxidizing 5hmC of the present invention and a bisulfite sequencing method, FIG. 10B shows the results of a ruthenium method and a bisulfite sequencing method, and FIG. 10C shows the results of a TAB-seq method and a bisulfite sequencing method.

### DESCRIPTION OF EMBODIMENTS

### 1. Oxidizing Agent for 5-Hydroxymethylcytosine

### 1-1. Outline

A first aspect of the present invention is an oxidizing agent for 5-hydroxymethylcytosine (an oxidizing agent for 5hmC). The oxidizing agent of the present invention is characterized in that it oxidizes only a hydroxy group in an allyl alcohol structure contained in 5hmC and converts it to 5fC. Using the oxidizing agent of the present invention, 5hmC that may be contained in a biological sample can be selectively oxidized into 5fC at low costs, without occurrence of side reactions such as DNA degradation. Accordingly, the oxidizing agent for 5hmC of the present invention can also be understood to be a conversion agent for converting 5hmC to 5fC.

### 1-2. Definition of Terms

The terms frequently used in the present description are defined as follows.

“5-Hydroxymethylcytosine” (5hmC) is a modification of cytosine that is one of pyrimidine bases, and it is generated by hydroxylation of the methyl group of 5mC by the catalytic activity of an enzyme such as TET in vivo, as mentioned above. 5-Hydroxymethylcytosine is a starting substance in a DNA demethylation reaction, and therefore, it can be an indicator of DNA demethylation. 5hmC is a target molecule that can be a target to be selectively oxidized by the oxidizing agent for 5hmC of the present invention.

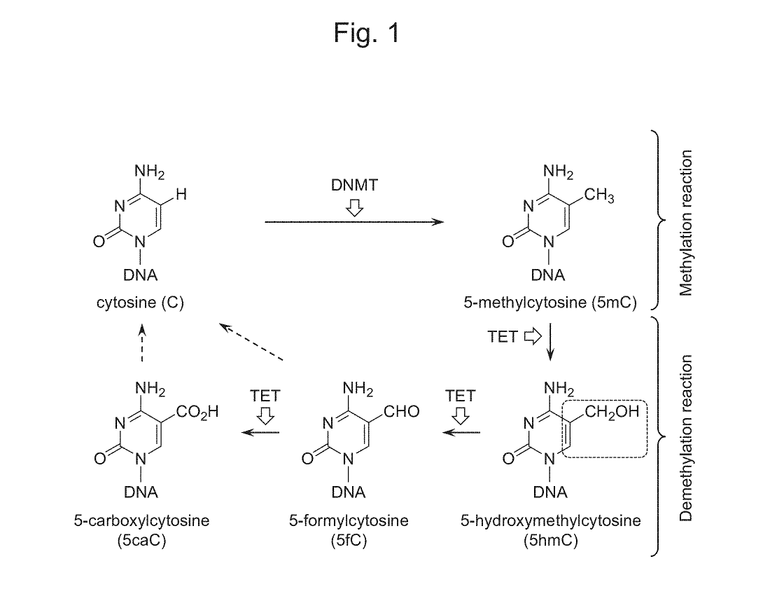

“Allyl alcohol structure” is an intramolecular structure including the basic backbone of allyl alcohol (2-propen-1-ol). The allyl alcohol structure is a structure that is extremely rarely present in biomolecules. As shown in FIG. 1, 5 hmC that is the target molecule of the present invention has an allyl alcohol structure in the molecule thereof (the broken line frame in FIG. 1).

“Allyl alcohol” is known as unsaturated alcohol having the simplest structure. The oxidizing agent for 5hmC of the present invention selectively oxidizes allyl alcohol or a hydroxy group in the allyl alcohol structure. As mentioned above, since the allyl alcohol structure is extremely rarely present in biomolecules, the oxidizing agent for 5hmC of the present invention substantially can selectively oxidize only 5hmC.

“5-Formylcytosine” (5fC) is a modification of cytosine, and is generated by the oxidation of the hydroxy group in the allyl alcohol structure of 5hmC. 5-Formylcytosine is known as an intermediate product following 5hmC in a DNA demethylation reaction in vivo (FIG. 1). While it is difficult to directly detect 5hmC, various methods of detecting 5fC, including a bisulfite method as a typical example, have been established, and it is easy to directly detect 5fC. Therefore, as a method of chemically detecting 5hmC, an indirect detection method, which oxidizes 5hmC into 5fC and then detects the 5fC, as with a ruthenium method, is used. Also in a fifth aspect of the present invention, as mentioned later, this principle is applied.

“Biological sample” is an organism-derived sample. In the present description, the biological sample can be a sample possibly comprising DNA as a test substance in the method of analyzing 5-hydroxymethylcytosine of the after-mentioned fifth aspect, and preferably, demethylated DNA. Specific examples of the biological sample include cells (including tissues and organs) and a body fluid possibly comprising cells. In the present description, the “body fluid” means a liquid sample directly collected from an individual organism. Examples of the body fluid include blood (including whole blood, serum, plasma and interstitial fluid), lymph fluid, cerebrospinal fluid, nerve root fluid, synovial fluid, lacrimal fluid, nasal discharge, saliva, urine, sweat, milk, sputum, vaginal fluid, semen, pleural effusion, and ascites. The biological sample used in the present invention may be cells of any tissue or organ, or may also be any of the above-described body fluids. In the case of using a biological sample collected from a living body, namely, from a living individual organism, such a biological sample is preferably cells or a body fluid, which causes low invasiveness to an individual organism upon collection thereof. Examples of the cells used as such a biological sample include epithelial cells constituting oral mucosa, nasal mucosa, vaginal mucosa and intestinal mucosa, hair matrix cells, and keratinocytes. Examples of the body fluid used as such a biological sample include blood, saliva, nasal discharge, sputum, vaginal fluid, and semen.

In the present description, 5hmC as a direct target may be present in any state, as long as it retains an allyl alcohol structure. Examples of the state of 5hmC include a free state as a pyrimidine base, a state in which 5hmC binds to a peptide or an organic polymer, and the state of a base constituting a nucleic acid. In general, 5hmC is present as a base constituting a nucleotide (including ribonucleotide and deoxyribonucleotide) that is a constituting unit of a nucleic acid such as DNA or RNA, or a base constituting a nucleoside (including ribonucleoside and deoxyribonucleoside) that constitutes the nucleotide. When 5hmC is contained in a biological sample, although a nucleic acid containing 5hmC is derived from the nature, the nucleic acid as a target of the present invention is not necessarily derived from the nature, but it also includes an artificially synthesized nucleic acid. For example, the nucleic acid as a target of the present invention may be chemically synthesized DNA containing 5hmC.

### 1-3. Configuration

### 1-3-1. Components

The oxidizing agent for 5hmC of the present invention is composed of a nitroxyl radical molecule and a copper salt or a copper complex, and/or a nitroxyl radical molecule-copper complex. All of these components are available at relatively low costs. In addition, a reaction promoter(s) can also be added as a component(s) of the oxidizing agent of the present invention, as necessary.

In the present description, the “nitroxyl radical molecule” means a compound having one or more nitroxyl radicals (—NO.) in the molecule thereof. The nitroxyl radical is also referred to as a “nitroxide radical,” and it is a radical molecule containing a nitrogen atom as a centered radical. The nitroxyl radical is known as a radical, which has high reactivity with active oxygen or an oxidation-reduction substance, but is relatively stable as a result of a resonance structure between radical species on the oxygen atom and cation radical species on the central nitrogen atom. The nitroxyl radical molecule is a component essential for the oxidizing agent for 5hmC of the present invention.

The nitroxyl radical molecule used in the present invention may be either an organic nitroxyl radical molecule or an inorganic nitroxyl radical molecule. It is preferably an organic nitroxyl radical molecule.

Examples of the type of the nitroxyl radical molecule include, but are not limited to, 2,2,6,6-tetramethylpiperidine-1-oxyl (hereinafter often referred to as “TEMPO”), 3-carbamoyl-2,2,5,5-tetramethyl-3-pyrrolin-1-yloxy (hereinafter often referred to as “3-Carbamoyl-PROXYL”), 2-azaadamantane-N-oxyl (hereinafter often referred to as “AZADO”), 9-azabicyclo[3.3.1]nonane-N′-oxyl (hereinafter often referred to as “ABNO”), and a derivative thereof.

In the present description, the “copper salt” means a compound in which a copper (Cu) ion as a cation binds to an acid-derived anion. The copper salt may be either an inorganic compound or an organic compound, but it is generally an inorganic compound. The copper ion may be either a monovalent copper ion (Cu⁺: copper (I) ion) or a divalent copper ion (Cu²⁺: copper (IT) ion), but it is preferably a divalent copper ion. Specific examples of the copper salt having a copper (I) ion include copper chloride (I) (CuCl), copper perchlorate (I) (CuClO₄), copper oxide (I) (Cu₂O), and copper (I) triflate. In addition, specific examples of the copper salt having a copper (II) ion include copper chloride (II) (CuCl₂), copper perchlorate (I) (Cu(ClO₄)₂), copper oxide (II) (CuO), copper (II) triflate, copper sulfide (CuS), and copper sulfate (CuSO₄). In the case of using a copper salt having a copper chloride (I) ion, copper (II) ions may be generated by nitroxyl radical molecules or oxidizing agents in the reaction system. At least one of the “copper salt” and the below-mentioned “copper complex” becomes a component that is essential for the oxidizing agent for 5hmC of the present invention.

In the present description, the “copper complex” means a complex consisting of a copper ion and a ligand bound thereto. The ligand bound to a copper ion is not particularly limited. An example of the copper complex is a copper-tetraamminecopper(II) complex comprising ammonia as a ligand. The below-mentioned nitroxyl radical molecule-copper complex is one of the aforementioned copper complexes.

In the present description, the “nitroxyl radical molecule-copper complex” means a complex formed by binding a nitroxyl radical molecule as a ligand to a copper ion, which is a compound directly contributing to oxidation of an allyl alcohol group in 5hmC. The nitroxyl radical molecule-copper complex is formed by mixing the aforementioned nitroxyl radical molecule with a copper salt or a copper complex in a solution. That is to say, the nitroxyl radical molecule-copper complex is considered to be a compound in which the reaction has proceeded one step from a nitroxyl radical molecule and a copper salt or a copper complex in the oxidizing agent for 5hmC. Accordingly, the nitroxyl radical molecule-copper complex alone can function as an oxidizing agent for 5hmC of the present invention.

The oxidizing agent for 5hmC of the present invention may be: a combination of a nitroxyl radical molecule and a copper salt or a copper complex; a nitroxyl radical molecule-copper complex alone; or a mixture of a combination of a nitroxyl radical molecule and a copper salt or a copper complex, and a nitroxyl radical molecule-copper complex.

In the present description, the “reaction promoter” is an optional component constituting the oxidizing agent for 5hmC of the present invention, which is a compound having the function of an oxidation auxiliary agent for promoting the oxidation of the hydroxy group in the allyl alcohol structure by the nitroxyl radical molecule-copper complex. The type of the reaction promoter is not particularly limited, as long as it has the above-described function. Examples of the reaction promoter include pyridine, bipyridine, phenanthroline, ethylenediamine, propanediamine, imidazole, and a derivative thereof. The reaction promoter constituting the oxidizing agent for 5hmC may be a single type of agent, or a combination of multiple types of agents.

### 1-3-2. Form

The form of the oxidizing agent for 5hmC of the present invention is not particularly limited. It may be either a solid form (including powders, granules, and gel), or a liquid form. It is also possible that the components each have a different form. For example, it is possible that the nitroxyl radical molecule is in a powder form and the copper salt is in an aqueous solution form containing the copper salt in an ionic state.

Moreover, all or a part of components can be separated as individual components. In this case, upon the oxidation reaction of 5hmC, all of the components are mixed with one another, so that the obtained mixture can exhibit the effects of the oxidizing agent for 5hmC.

On the other hand, individual components may be integrated in advance. Examples of the components, which may be integrated in advance, include a state in which the powders of nitroxyl radical molecules are mixed with the powders of copper salts, and the state of a mixed solution prepared by dissolving the nitroxyl radical molecules and the copper salts in a solution.

### 2. DNA Demethylation Analysis Reagent

### 2-1. Outline

A second aspect of the present invention is a DNA demethylation analysis reagent. The analysis reagent of the present invention comprises the oxidizing agent for 5hmC of the first aspect. According to the analysis reagent of the present invention, 5fC generated by the oxidation of 5hmC that is a starting substance of DNA demethylation and is also a target substance of the oxidizing agent for 5hmC is detected, so that a demethylation site generated on DNA can be identified.

### 2-2. Definition

In the present description, the term “analysis” is often used to mean indirect detection and/or identification. For example, the DNA demethylation analysis reagent of the present aspect is a reagent capable of indirectly detecting the presence or absence of demethylation on DNA via detection of 5fC generated by the oxidizing agent for 5hmC of the first aspect, and then indirectly identifying the demethylation site on the DNA as positional information of 5fC on the DNA. Moreover, the after-mentioned DNA demethylation analysis kit of a third aspect is a kit capable of indirectly detecting the presence or absence of demethylation of DNA and indirectly identifying the demethylation site on the DNA. Furthermore, the method of analyzing 5hmC of a fifth aspect comprises indirectly detecting the presence or absence of 5hmC on DNA via detection of 5fC, and/or indirectly identifying the positional information of 5hmC on the DNA as positional information of 5fC, when the DNA contains 5hmC.

### 2-3. Configuration

### 2-3-1. Components

The DNA demethylation analysis reagent of the present invention comprises, as essential components, an oxidizing agent for 5hmC, and a detection reagent for 5fC detecting 5fC generated by oxidation of 5hmC.

(Oxidizing Agent for 5hmC)

The present oxidizing agent for 5hmC is the oxidizing agent for 5hmC described in the aforementioned first aspect. Accordingly, since the specific configuration of the oxidizing agent for 5hmC is equivalent to the first aspect, the explanation thereof is omitted herein.

### (Detection Reagent for 5fC)

The specific configuration of the detection reagent for 5fC is not limited, as long as the detection reagent is a reagent used in a method capable of detecting 5fC on DNA. An example of the detection reagent for 5fC is a bisulfite used in a bisulfite sequencing method. Specific examples of the bisulfite include sodium bisulfite (NaHSO₃), potassium bisulfite (KHSO₃), and ammonium bisulfite ((NH₄)HSO₄).

### 2-3-2. Form

The form of each component in the DNA demethylation analysis reagent of the present invention is not particularly limited. The form of the component may be either a solid form (including powders, granules, and gel), or a liquid form. It is also possible that the components each have a different form. For example, it is possible that the oxidizing agent for 5hmC is in a liquid form and the detection reagent for 5fC is in a powder form.

In principle, individual components in the DNA demethylation analysis reagent of the present invention are separated from one another. For example, the oxidizing agent for 5hmC and the detection reagent for 5fC are individually packaged, and when 5hmC on DNA is detected, individual components may be used individually in an appropriate reaction step in which they are used.

### 3. DNA Demethylation Analysis Kit

### 3-1. Outline

A third aspect of the present invention is a DNA demethylation analysis kit. Reagents necessary for analyzing DNA demethylation and the like are incorporated into the kit of the present invention. Using the kit of the present invention, it becomes possible to simply and quickly carry out the analysis of DNA demethylation.

### 3-2. Configuration

The DNA demethylation analysis kit of the present invention comprises, as an essential component, a DNA demethylation analysis reagent. The present kit also comprises, as necessary, an optional reagent, a reaction vessel, an instruction manual, and the like, which are necessary for a DNA demethylation reaction. Hereafter, the above-described components are described.

The “DNA demethylation analysis reagent” is the DNA demethylation analysis reagent described in the aforementioned second aspect, and the specific configuration of the present DNA demethylation analysis reagent is as described in the second aspect, and thus, the explanation thereof is omitted herein.

The “optionalreagent” is an optional component in the kit of the present invention, and such an optional reagent can be appropriately selected, as necessary, and can be added to the kit. Examples of the optional reagent include, but are not limited to, a buffer and a labeling reagent. The “buffer” is a solvent or a solution used in each step to perform an appropriate treatment, such as separation and purification of DNA, or to smoothly drive reactions. The composition or pH of the buffer may be determined, as appropriate. The “labeling reagent” is a reagent used to label nucleic acids or bases, and a labeling reagent known in the present technical field can be used herein. Examples of the labeling reagent include fluorescent dyes (e.g., fluorescamine and a derivative thereof, rhodamine and a derivative thereof, FITC, cy3, cy5, FAM, HEX, and VIC), quencher substances (TAMRA, DABCYL, BHQ-1, BHQ-2, and BHQ-3), modifications such as biotin, avidin or streptavidin and magnetic beads, and radioisotopes (e.g., ³²P, ³³P, and ³⁵S).

The “reaction vessel” is a vessel used to treat a sample or perform a reaction, when DNA demethylation analysis is conducted. The size, shape, and volume of the reaction vessel are not particularly limited, as long as the reaction vessel is used to perform a treatment or a reaction on a sample. Examples of the reaction vessel include a 50-mL tube, a 1.5-mL tube, a 0.2-mL tube, and a 96-well microtiter plate. The material of the reaction vessel is not particularly limited, either, as long as it does not affect the reactions of the DNA demethylation analysis. Examples of the material of the reaction vessel include a plastic such as polypropylene or polyethylene, a glass, a ceramic, and a metal. In addition, the present reaction vessel also includes peripheral equipment such as a filter or a chip, although these are not vessels for retaining content.

The “instruction manual” describes appropriate reaction conditions (a dose, a reaction time, a reaction temperature, etc.) for carrying out DNA demethylation analysis using the sample included in the kit of the present invention.

### 4. Method of Oxidizing 5-Hydroxymethylcytosine

### 4-1. Outline

A fourth aspect of the present invention is a method of oxidizing 5hmC (5hmC oxidation method). The oxidation method of the present invention is a method of using the oxidizing agent for 5hmC described in the first aspect, in which 5hmC is converted to 5fC by selectively oxidizing the hydroxy group in the allyl alcohol structure contained in 5hmC.

### 4-2. Method

The oxidation method of the present invention comprises, as essential steps, a “mixing step” and an “oxidation step.” Hereafter, individual steps are specifically described.

### (1) Mixing Step

The “mixing step” is a step of mixing a test substance with the oxidizing agent for 5hmC described in the first aspect in a reaction solution.

The “test substance” is a substance possibly comprising 5hmC, which is subjected to the present method. In general, the test substance is a nucleic acid derived from a living body as a test subject, in particular, DNA and more preferably, genomic DNA, but the test substance is not limited thereto. DNA is generally present in the form of double strands in vivo. When DNA is subjected to the present method, however, it must be in a state in which 5hmC is not paired with another base. Accordingly, when the test substance used in the present step is DNA, the DNA is, in principle, a single strand.

The “test subject” is an individual organism, biological tissues (including organs), or cells, which are subjected to the method of analyzing 5hmC of the present invention. The organism species may be any of an animal, a plant, fungus, and bacteria. The test subject of the present invention is preferably an animal, and more preferably a vertebrate, but is not limited thereto. The test subject of the present invention is preferably a mammal, and particularly preferably a human.

The term “mixing” is used to mean that two or more substances each having different properties are mixed with one another, such that they come into contact with one another. In the present step, such mixing is carried out in a reaction solution, namely, in a liquid.

A nitroxyl radical molecule, a copper salt or a copper complex, and a nitroxyl radical molecule-copper complex, which constitute the oxidizing agent for 5hmC, are desirably each comprised in an amount of 1 to 10000 equivalents, and preferably 10 to 1000 equivalents, in the reaction solution.

When the oxidizing agent for 5hmC described in the first aspect consists of two or more components, for example, a nitroxyl radical molecule and a copper salt or a copper complex, the order of mixing the components of the oxidizing agent for 5hmC with a test substance is not determined in the present step. That is, a test substance, a nitroxyl radical molecule, and a copper salt or a copper complex may be added to and mixed with one another in this order. Otherwise, a test substance, a copper salt or a copper complex, and a nitroxyl radical molecule may also be added to and mixed with one another in this order. Alternatively, it is also possible that a nitroxyl radical molecule, a copper salt or a copper complex, and a test substance are added to and mixed with one another in this order, or that a test substance, a nitroxyl radical molecule, and a copper salt or a copper complex are simultaneously added to and mixed with one another.

In the present step, the mixing method is not particularly limited. The reaction solution may be stirred using a stirrer or a stirring bar, or it may also be mixed by inverting, rotating or shaking the reaction tank.

### (2) Oxidation Step

The “oxidation step” is a step of incubating the reaction solution obtained after the mixing step at a temperature of 4° C. to 90° C., preferably 15° C. to 70° C., and more preferably 25° C. to 60° C., for 1 to 100 hours, preferably for 5 to 50 hours, and more preferably for 10 to 24 hours, so that the hydroxy group in an allyl alcohol structure comprised in 5hmC is oxidized.

During the present step, the reaction solution may be in a stationary state, or may be stirred to unify temperature distribution in the solution. When the present step is carried out in a place in which the surrounding temperature is constant, such as the inside of an incubator, in general, it is sufficient to carry out the step in a stationary state.

The reaction solution obtained after the present step may contain DNA containing 5fC, which is generated by selectively oxidizing the hydroxy group in the allyl alcohol structure in 5hmC, using the oxidizing agent for 5hmC of the first aspect.

### 5. Method of Analyzing 5-Hydroxymethylcytosine

### 5-1. Outline

A fifth aspect of the present invention is a method of analyzing 5-hydroxymethylcytosine (a method of analyzing 5hmC). The method of the present invention is a method of indirectly detecting 5hmC, in which 5hmC that has been generated on DNA in a biological sample by a demethylation reaction is converted to 5fC by the above-described method of oxidizing 5hmC of the fourth aspect, and the the 5fC is detected. According to the present method, a demethylation site on DNA can be identified.

### 5-2. Method

The analysis method of the present invention comprises, as essential steps, a “mixing step,” an “oxidation step,” and a “detection step.” This method also comprises, as optional steps, a “DNA extraction step” and a “hydrogen bond cleavage step.” Hereafter, individual steps are specifically described.

### (1) DNA Extraction Step

The “DNA extraction step” is a step of extracting DNA, in particular, high molecular weight DNA such as genomic DNA, from a biological sample, before the after-mentioned hydrogen bond cleavage step. The method of extracting DNA is not particularly limited, as long as high molecular weight DNA can be extracted from a biological sample. Examples of the DNA extraction method include a method of proteolytic digestion of a biological sample by proteinase K followed by treatment with phenol and chloroform solution, and a hot-shot method. With regard to a specific method of extracting high molecular weight DNA, the method described in Green, M. R. and Sambrook, J., 2012, Molecular Cloning: A Laboratory Manual Fourth Ed., Cold Spring Harbor Laboratory Press, Cold Spring Harbor, N.Y. may be referred to. Kits of extracting high molecular weight DNA such as genomic DNA from a biological sample are commercially available from individual manufacturers, and such kits can also be utilized. In such a case, the specific extraction method may be carried out in accordance with the instruction manual included with the kit.

### (2) Hydrogen Bond Cleavage Step

The “hydrogen bond cleavage step” is a step of cleaving a hydrogen bond between bases, when the DNA is double-stranded DNA, to denature the DNA to single-stranded DNA.

Organism-derived DNA is generally in the state of double stands. When 5hmC in DNA is oxidized using the method of oxidizing 5hmC of the fourth aspect, however, the DNA must be in a single-stranded state without a self-folding structure, as mentioned above. Hence, in the present step, the hydrogen bond in double-stranded DNA is cleaved to obtain single-stranded DNA. However, when the DNA obtained by the above-described DNA extraction step is single-stranded DNA, the present step is not necessary.

The method of cleaving a hydrogen bond is not particularly limited, as long as it is a method that does not affect other chemical bonds in DNA. A common method in the present technical field may be applied. Examples of such a cleavage method include an alkali treatment for cleaving the bond with strong alkali such as NaOH, a high temperature treatment, and a DNA helicase treatment. With regard to the method of cleaving a hydrogen bond, the method described above in Green, M. R. and Sambrook, J. (2012) can also be referred to.

### (3) Mixing Step

The “mixing step” is a step equivalent to the mixing step in the above-described method of oxidizing 5hmC of the fourth aspect. The specific explanation of the step is the same as that described in the fourth aspect, and thus, the explanation thereof is omitted herein.

### (4) Oxidation Step

The “oxidation step” is a step equivalent to the oxidation step in the above-described method of oxidizing 5hmC of the fourth aspect, as in the case of the aforementioned mixing step. The specific explanation of the step is the same as that described in the fourth aspect, and thus, the explanation thereof is omitted herein.

### (5) Detection Step

The “detection step” is a step of detecting 5fC that has been generated in the above-described oxidation step. The method of detecting 5fC may be a method known in the present technical field, and thus, it is not particularly limited. Examples of the detection method include an identification method by an enzyme treatment, an identification method by a chemical decomposition reaction, an identification method using a labeling regent, and a bisulfite sequencing method.

An example of the identification method by an enzyme treatment is a method of using an enzyme such as alkaline phosphatase (AP) or nuclease P1 (P1). In this method, a nucleic acid obtained after the oxidation step is decomposed to a nucleoside using the above-described enzyme. Upon decomposition, phosphodiesterase is further added, so that the nucleic acid can be more efficiently decomposed to a nucleoside. Subsequently, the obtained nucleoside is analyzed by HPLC, TLC, etc., so as to detect 5fC.

An example of the identification method by a chemical decomposition reaction is a reaction of cleaving 5fC-containing DNA, using piperidine. When 5fC-containing DNA is treated with piperidine, a cleavage reaction takes place on the 3′ side of 5fC, and then, on the 5′ side thereof. On the other hand, in the case of 5hmC, cytosine, or 5-methylcytosine, such a cleavage reaction does not take place. After the reaction, DNA is separated by gel electrophoresis, etc, etc, and the presence or absence of cleavage is then confirmed based on a difference in DNA sizes, so that 5fC can be detected. At that time, if the terminal portion of DNA has been labeled with a suitable labeling agent before the decomposition, detection becomes easy, and thus, it is convenient. In addition, when the accurate position of 5fC in the nucleotide sequence of DNA is to be identified, both the nucleotide sequence of DNA before the decomposition reaction and that of DNA after the decomposition reaction may be determined, and the two sequences may be then compared with each other.

In the identification method of using a labeling reagent, 5hmC is labeled with a labeling reagent having a hydrazide group, such as biotin hydrazide or fluorescein hydrazide, and thereafter, 5fC can be detected based on the properties of the labeling reagent.

The bisulfite sequencing method is a method of utilizing the conversion of non-methylated cytosine to uracil (U) by a bisulfite treatment, and this method is one of the most common methods of detecting 5fC. Since 5mC and 5hmC are not converted to the aforementioned “U” after the bisulfite treatment, if a nucleic acid amplification reaction such as PCR is carried out on the treated DNA, the position will be “C”. On the other hand, since C, 5fC and 5caC are converted to “U” after the bisulfite treatment, the position is changed to thymine (T) after the nucleic acid amplification reaction. Herein, if DNA is treated by the method of oxidizing 5hmC of the fourth aspect of the present invention before performing a bisulfite treatment, 5hmC is converted to 5fC. Thus, 5hmC is converted to “T”, after the subsequent bisulfite treatment and nucleic acid amplification reaction. That is to say, among C, 5mC, 5hmC, 5fC and 5caC, only in the case of 5hmC, the base in the nucleic acid amplification reaction changes between after the bisulfite treatment, and after a combination of the bisulfite treatment and the method of oxidizing 5hmC of the present invention. Accordingly, the presence or absence of the bisulfite treatment is combined with the presence or absence of the method of oxidizing 5hmC of the present invention, and each combination is performed for DNA. The obtained DNAs are each subjected to nucleic acid amplification and nucleotide sequencing. The position of 5hmC can be easily identified by comparison of the sequences.

### EXAMPLES

### Example 1

### (Purpose)

The reaction in which 5hmC in DNA is specifically oxidized into 5fC by the method of oxidizing 5hmC of the present invention was examined by performing a cleavage reaction using piperidine.

### (Method)

As target DNA, nucleotides consisting of the nucleotide sequence shown in SEQ ID NO: 3 (5′-Fluo-aaaaaaaaagxgaaaaaa-3′; x=5hmC) were synthesized (the synthesis was outsourced to GeneDesign, Inc.). The 5′-terminus of this target DNA was fluorescently labeled with fluorescein.

Thereafter, 2 μL of 100 μM target DNA (15 mer), 55 μL of MilliQ, 10 μL of 50 mM Cu(ClO₄)₂, 10 μL of 50 mM TEMPO/acetonitrile solution, 10 μL of 50 mM NaOH, and 15 μL of 50 mM bipyridine/acetonitrile solution were placed in a 1.5-mL sample tube and were then mixed with one another (mixing step). Herein, TEMPO and Cu(ClO₄)₂ corresponded respectively to a nitroxyl radical molecule and a copper salt, which constitute the oxidizing agent for 5hmC of the present invention. As a control, a sample was prepared without using either a TEMPO/acetonitrile solution or Cu(ClO₄)₂. After the mixing, the mixture was left at 50° C. for 44.5 hours (oxidation step).

Subsequently, the reaction solution was transferred to a Bio-Spin Column (BioRad), and was then centrifuged at 1000 rpm for 4 minutes to remove Cu. After that, 8.9 μL of piperidine was added to the residue. The concentration of piperidine at this time was 15%. Besides, as a control, a sample was prepared without addition of piperidine. The solution was incubated at 90° C. for 2 hours, and then concentrated by drying for 40 minutes using a vacuum condenser (EYELA; centrifugal evaporator CVE3100), so that the solvent was removed.

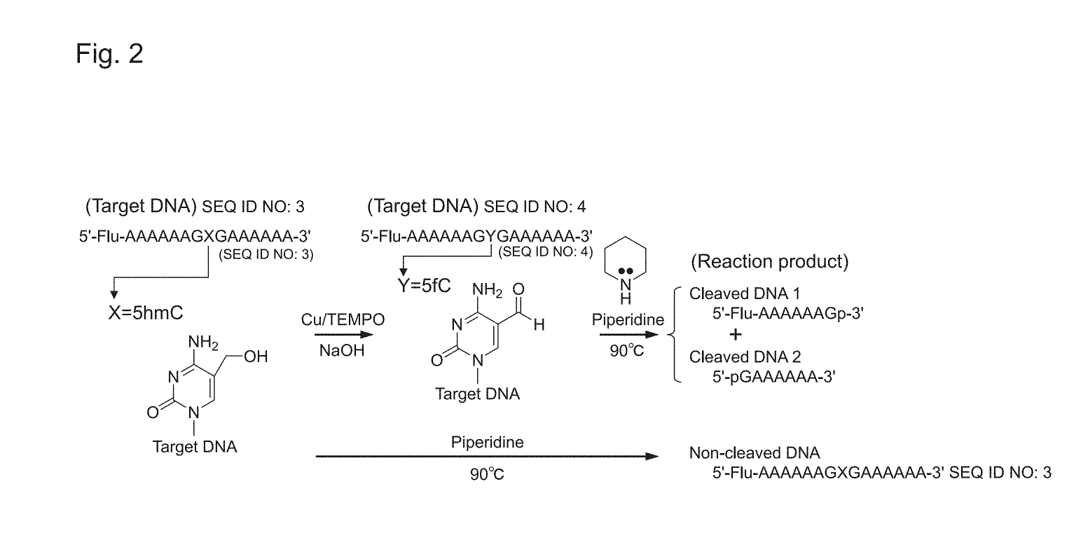

When 5hmC represented by x in the target DNA consisting of the nucleotide sequence shown in SEQ ID NO: 3 was converted to 5fC by the oxidizing agent for 5hmC of the present invention, the 3′ side of 5fC was first cleaved by a piperidine treatment, and then, the 5′ side of 5fC was also cleaved. As a result, 5fC was removed, and two cleaved DNA fragments each consisting of 7 mer were generated (see FIG. 2).

The obtained nucleic acid was separated by 20% acrylamide gel electrophoresis. As markers for DNA size, untreated 15-mer target DNA and 7-mer DNA corresponding to the cleaved DNA were electrophoresed at the same time. After the electrophoresis, DNA was detected using fluorescein, and the fluorescence intensity of a DNA band was analyzed using the image processing software ImageJ (http://imagej.nih.gov/ij/).

### (Results)

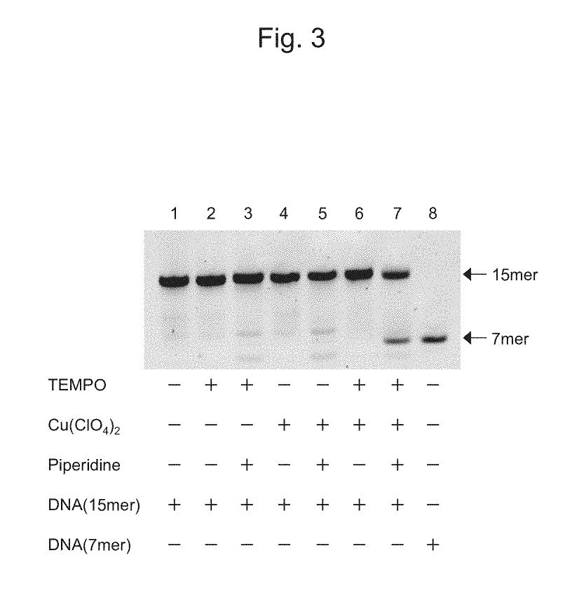

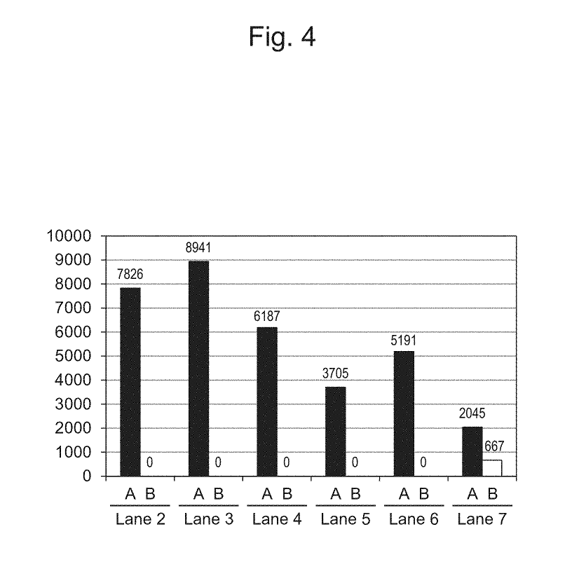

FIG. 3 shows the electrophoresis-gel, and FIG. 4 shows the fluorescence intensity of DNA bands in FIG. 3 as a graph. Only in Lane 7 loaded with a sample to which TEMPO and Cu(ClO₄)₂ were added, a 7-mer DNA band was observed. On the other hand, in Lane 6 loaded with a sample to which TEMPO and Cu(ClO₄)₂ were added, but to which piperidine was not added, such a 7-mer DNA band could not be observed. These results suggest that, with regard to the 7-mer DNA in Lane 7, 5hmC in target DNA was converted to 5fC by TEMPO and Cu(ClO₄)₂ that constituted the oxidizing agent for 5hmC of the present invention, and as a result, it was cleaved by piperidine.

### Example 2

### (Purpose)

Specific oxidation of deoxy-5-hydroxymethylcytidine by the oxidizing agent for 5hmC of the present invention was examined.

### (Method)

As a nucleoside to be examined, deoxy-5-hydroxymethylcytidine (d5hmC) was used, and as a control nucleoside, deoxy-5-methylcytidine (d5mC) was used. A solution containing 17 μM nucleoside, 4.3 mM Cu(ClO₄)₂, 4.3 mM TEMPO, 4.3 mM NaOH, and 6.5 mM bipyridine was left at room temperature for 1 to 3 days. Thereafter, the solution was 5-fold diluted with water, and was then analyzed by HPLC. In the HPLC, a reverse phase column (Thermo BioBasic-18, 180×4.6) was used, elution was carried out at a flow rate of 1 mL/min, and signal detection was then carried out using 254 nm light. As an eluent, an acetonitrile solution containing 2%-10% triethylammonium acetate was used.

### (Results)

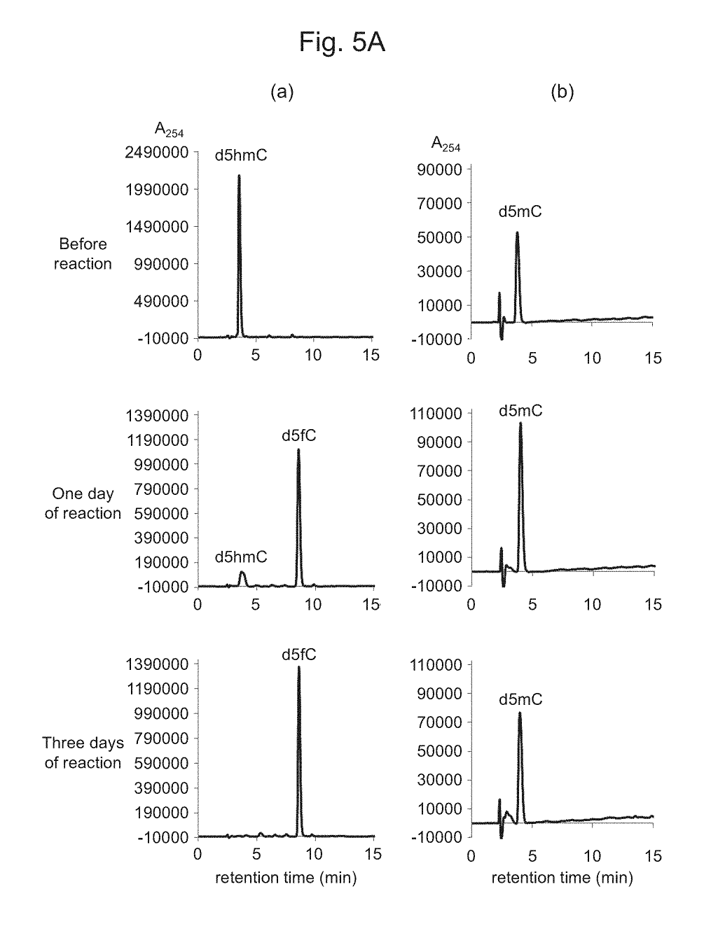

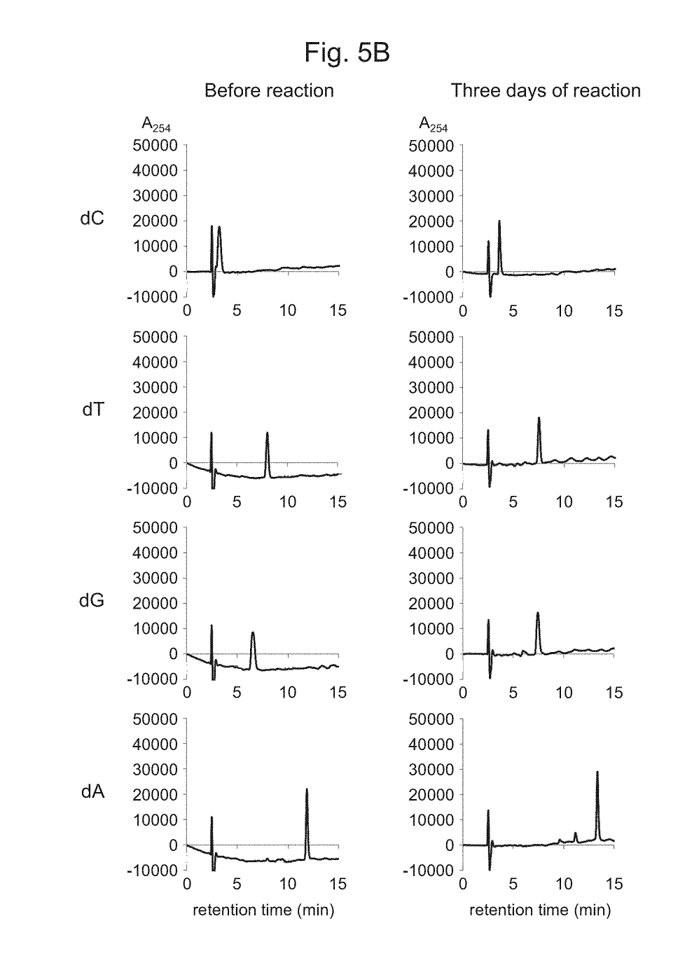

The results of the present example are shown in FIG. 5.

FIG. 5A shows the results obtained by treating (a) d5hmC and (b) d5mC with the oxidizing agent for 5hmC of the first aspect. The HPLC analysis results before the oxidation reaction, after one day of reaction, and after three days of reaction are shown.

As shown in FIG. 5A(a), d5hmC decreased over time after the reaction started, and completely disappeared after three days of reaction. In contrast, deoxy-5-formylcytidine (d5fC) increased over time after the reaction started, and d5fC completely replaced d5hmC after three days of reaction. These results suggest: that d5hmC was oxidized into d5fC by the oxidizing agent for 5hmC of the first aspect; and namely that only the hydroxy group in the methyl alcohol structure in d5hmC was specifically oxidized, whereas the alcohol structure of a ribose portion was not oxidized.

On the other hand, as shown in FIG. 5A(b), in the case of d5mC, almost no change was found in the peak patterns of HPLC before and after the reaction, suggesting that d5mC was not affected by the oxidizing agent for 5hmC of the first aspect.

FIG. 5B shows the results obtained by treating deoxycytidine (dC), deoxythymidine (dT), deoxyguanosine (dG), and deoxyadenosine (dA) with the oxidizing agent for 5hmC of the first aspect. The HPLC analysis results before the oxidation reaction and after three days of reaction are shown. For all of the deoxynucleosides, almost no change was found in the peak patterns of HPLC before and after the reaction. These results suggest that major deoxynucleosides other than d5hmC are not oxidized by the oxidizing agent for 5hmC of the present invention. Accordingly, it was demonstrated that the oxidizing agent for 5hmC of the present invention can specifically oxidize d5hmC in DNA.

### Example 3

### (Purpose)

At present, as a method of detecting 5hmC, a method of using a ruthenate is most common. However, this method is problematic in terms of side effects, such that it destabilizes the DNA structure and causes non-specific degradation. Hence, in the present example, the influence of the oxidizing agent for 5hmC of the present invention on the DNA structure, and the related presence or absence of the non-specific degradation of DNA, were examined.

### (Method)

As target DNA, the 15-mer DNA consisting of the nucleotide sequence shown in SEQ ID NO: 3, which was used in Example 1, was used.

### (1) Method of Oxidizing 5hmC of the Present Invention:

2 μL of 100 μM target DNA, 55 μL of MilliQ, 10 μL of 50 mM Cu(ClO₄)₂, 10 μL of 50 mM TEMPO/acetonitrile solution, 10 μL of 50 mM NaOH, and 15 μL of 50 mM bipyridine/acetonitrile solution were placed in a 1.5-mL sample tube and were then mixed with one another. Thereafter, the obtained mixture was left at room temperature for 1 day.

### (2) Control:

2 μL of 100 μM target DNA and 100 μL of MilliQ were placed in a 1.5-mL sample tube and were then mixed with each other. Thereafter, the obtained mixture was left at 0° C. for 1 hour.

### (3) Ruthenate Method:

Ruthenium experiment: 2 μL of 100 μM target DNA, 3 μL of 3 mM KRuO₄, and 97 μL of 50 mM NaOH solution were placed in a 1.5-mL sample tube and were then mixed with one another. Thereafter, the obtained mixture was left at 0° C. for 1 hour.

Subsequently, the solution was transferred to a Bio-Spin Column, and was then centrifuged at 1000 rpm for 4 minutes to remove Cu. Subsequently, DNA was recovered by an ethanol precipitation method, and the obtained DNA was then used as template DNA in real-time PCR. The PCR solution was recovered, and was then 1000-fold diluted. The diluted DNA was used as template DNA, and 100 μM primers (2 μL each) consisting of the nucleotide sequences shown in SEQ ID NO: 1 and SEQ ID NO: 2, 2 μL of 100 mM dNTP, 0.2 μL of KOD Dash DNA polymerase (TOYOBO), and 2 μL of 1000-fold diluted SYBR Green (BioRad) were mixed with one another to prepare a mixture. For PCR conditions, a cycle consisting of 95° C.-15 seconds, 55° C.-15 seconds, and 75° C.-15 seconds was repeated 50 times.

Based on the number of PCR cycles and the temporal changes of fluorescence intensity of SYBR Green, the degree of non-specific DNA degradation due to oxidization caused by the method of oxidizing 5hmC of the present invention and by the ruthenate method can be estimated. Specifically, using the number of cycles at the midpoint of the sigmoid curve obtained by PCR (2/50=25 cycles in the present example), and also using a calibration curve showing the correlation between the predetermined concentration and the number of cycles, the initial concentration before PCR, namely, the DNA concentration after the oxidation reaction can be clarified.

### (Results)

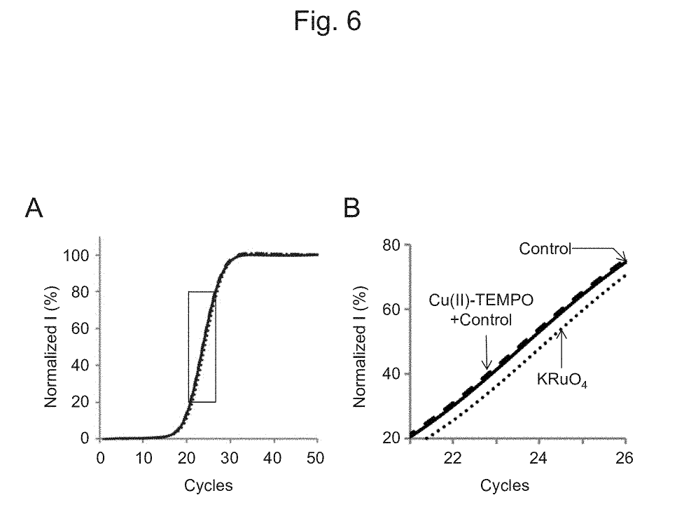

The results of Example 3 are shown in FIG. 6. FIG. 6B is an enlarged view of the frame in FIG. 6A. In terms of fluorescence intensity, the method of oxidizing 5hmC of the present invention shown with the broken line was almost overlapped with the control, and after the oxidation reaction, 90% of DNA remained without being damaged. In contrast, in the case of the conventional ruthenate method shown with the dotted line, fluorescence intensity was generally lower than that of the control, and only 38% of the original DNA remained. These results suggest that the method of oxidizing 5hmC of the present invention can selectively oxidize only the target 5hmC, while preventing non-specific DNA degradation.

All publications, patents and patent applications cited in the present description are incorporated herein by reference in their entirety.

### Example 4

### (Purpose)

The reproducibility of detection accuracy by the method of oxidizing 5hmC of the present invention was examined.

### (Method)

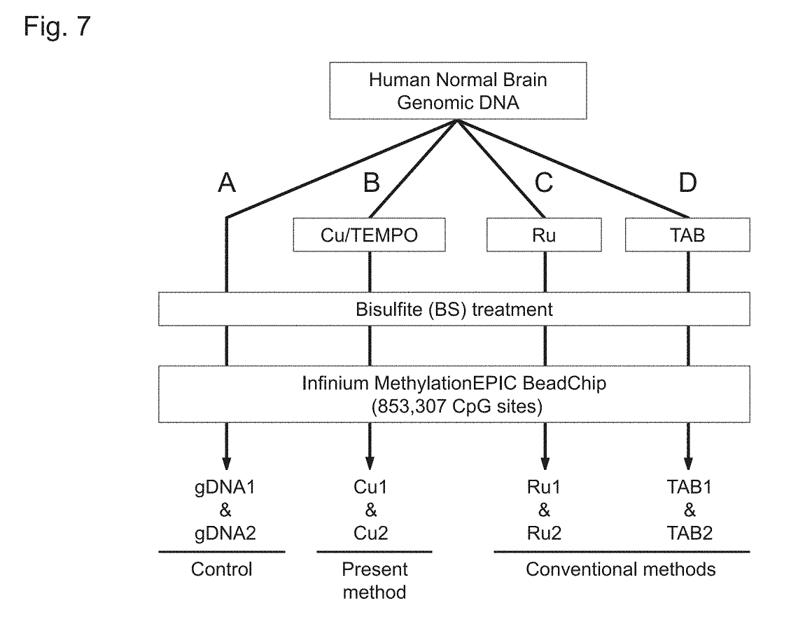

Using 2 mg of human brain-derived genomic DNA, various methods of detecting 5hmC shown in FIG. 7 were independently carried out twice under the same conditions. (B) Cu/TEMPO indicates the method of oxidizing 5hmC of the present invention, (C) Ru indicates the conventional ruthenium method, (D) TAB indicates the conventional TAB-Seq method, and A indicates a negative control, in which such a method of oxidizing 5hmC was not carried out.

The method of oxidizing 5hmC and the ruthenium method were carried out in accordance with the methods described in the aforementioned Examples 2 and 3. In addition, the TAB-Seq method was carried out using 5hmC TAB-Seq kit (WiseGene) in accordance with protocols included therewith.

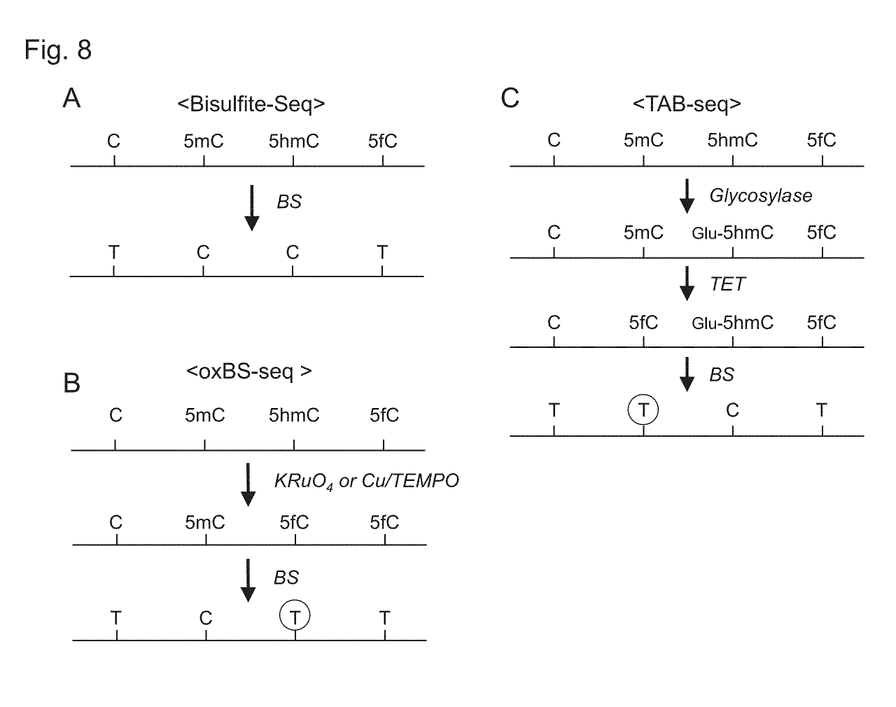

The “TAB-Seq method” is a method of glycosylating 5hmC on genomic DNA and then carboxylating only 5mC by a Tet1 treatment, as shown in FIG. 8C. In the conventional bisulfite sequencing method, genomic DNA was subjected to a bisulfite treatment (BS treatment), so that the methylation site on the genomic DNA could be detected, as shown in FIG. 8A. However, by this method, 5mC could not be distinguished from 5hmC in the methylation site. In the TAB-Seq method, 5hmC on genomic DNA was glycosylated, so that it could be protected from carboxylation by Tet1, and thus, only 5mC was converted to carboxylcytosine (caC). Accordingly, if the TAB-Seq method is applied to genomic DNA before the BS treatment, only 5mC is converted to uracil (U). As a result, 5mC is detected as T and 5hmC is detected as C by sequencing. As such, the bisulfite sequencing method and the TAB-Seq method are carried out on the same sample, so that 5mC can be distinguished from 5hmC on genomic DNA. On the other hand, as shown in FIG. 8B, the method of oxidizing 5hmC of the present invention and the ruthenium method are both methods of oxidizing 5hmC into 5fC by ruthenate such as potassium perruthenate or Cu/TEMPO, then converting the 5fC to uracil by a BS treatment, and then detecting it as T by sequencing.

The methylated states of methylation sites (CpG sites) on genomic DNA treated by each of the above-described methods of detecting 5hmC were examined using Infinium MethylationEPIC BeadChip (Illumina). Infinium MethylationEPIC BeadChip is a methylation array analysis kit capable of quantifying 850,000 or more methylation sites on total human genome at a resolution of 1 base. A specific method was carried out in accordance with protocols included with the kit.

### (Results)

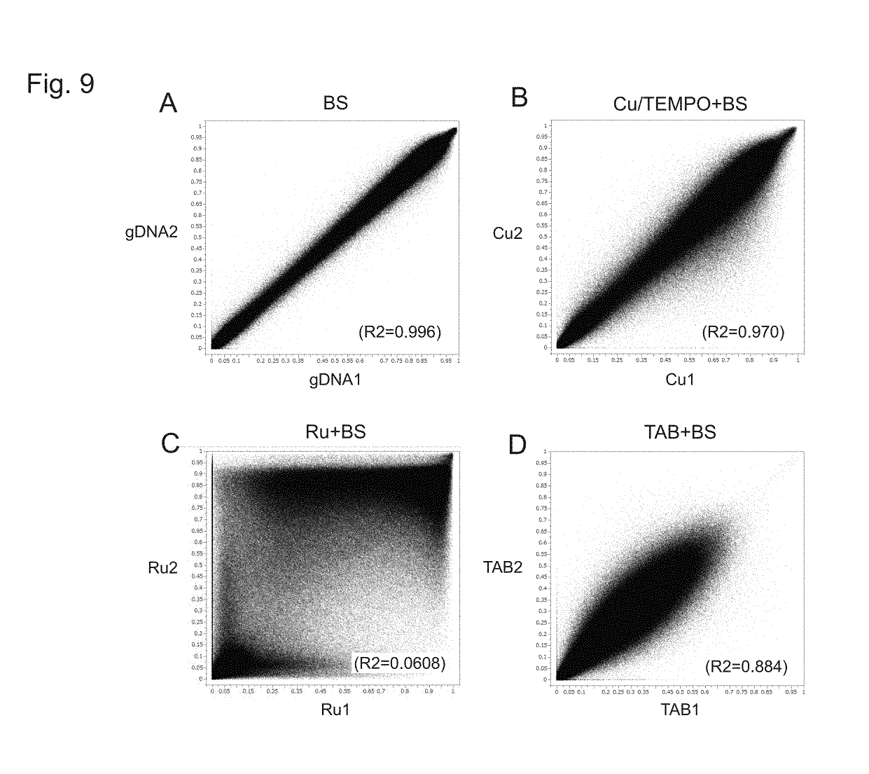

FIG. 9 shows the results detecting the reproducibility of each method for detecting 5hmC. The data are obtained by plotting, on the X-axis and Y-axis, the detection results of a single methylation site in two independent array analyses regarding 850,000 or more methylation sites on genomic DNA. FIG. 9A shows the results of a negative control with only the BS treatment, FIG. 9B shows the results of the method of oxidizing 5hmC of the present invention, FIG. 9C shows the results of the ruthenium method, and FIG. 9D shows the results of the TAB-seq method. As the methylated states in a single methylation site in the results of two analyses are similar to each other, the plots lie close to the diagonal line. Accordingly, a distribution of plots that is closer to the diagonal line suggests higher reproducibility of the method. As shown in FIG. 9, in the case of the method of oxidizing 5hmC of the present invention shown in FIG. 9B, individual plots fell mostly on the diagonal line, forming a relatively clear distribution along the diagonal line. These results show that the detection results obtained by the method of oxidizing 5hmC of the present invention have high reproducibility. On the other hand, in the case of the ruthenium method shown in FIG. 9C, the plots hardly showed a distribution pattern along the diagonal line, suggesting that this method has low reproducibility. In addition, in the TAB-Seq method, the plots showed a distribution pattern mostly along the diagonal line, but in a somewhat broad manner. Thus, it was suggested that the TAB-Seq method has lower reproducibility than the method of oxidizing 5hmC of the present invention.

### Example 5

### (Purpose)

The conversion rate of a methylation site on genomic DNA by each method of detecting 5hmC was examined.

### (Method)

Using the array analysis results of each method of detecting 5hmC obtained in Example 4, either one of the results of two experiments in each method was plotted on the X-axis, and the results from only the BS treatment as a negative control were plotted on the Y-axis. As shown in FIG. 8, in the case of the bisulfite sequencing method, 5mC and 5fmC on the genome are detected as “C”, whereas in the case of the method of oxidizing 5hmC of the present invention or the ruthenium method, 5mC is detected as “C”, and 5hmC is detected as “T”. Moreover, in the case of the TAB-seq method, 5mC is detected as “T”, and 5hmC is detected as “C”. In other words, the methylation site on genomic DNA, which has been appropriately converted by the treatment of each method of detecting 5hmC, would be different from the same methylation site processed only by the BS treatment. Such a difference would appear as a deviation from the diagonal line and thereby broaden the distribution from the diagonal line.

### (Results)

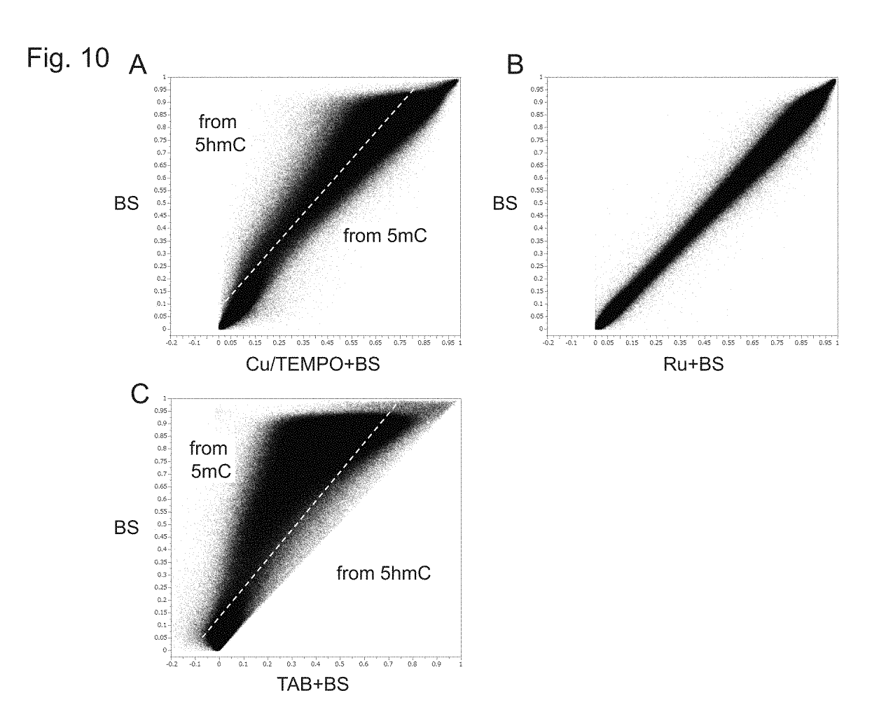

FIG. 10 shows the results of plotting. FIG. 10A shows the results of the method of oxidizing 5hmC of the present invention (X-axis) and only the BS treatment (Y-axis), FIG. 10B shows the results of the ruthenium method (X-axis) and only the BS treatment (Y-axis), and FIG. 10C shows the results of the TAB-seq method (X-axis) and only the BS treatment (Y-axis).

In FIG. 10A, plots above the broken line correspond to 5hmC-derived T, whereas in FIG. 10C, plots above the broken line correspond to 5mC-derived T. These results suggested that the methylation site on genomic DNA was appropriately converted by each treatment in the method of oxidizing 5hmC of the present invention and the TAB-seq method. In contrast, the ruthenium method did not show a deviation from the diagonal line, suggesting that a conversion reaction was not appropriately completed in the ruthenium method.

1. An oxidizing agent for 5-hydroxymethylcytosine, selected from the group consisting of:
(a) a nitroxyl radical molecule and a copper salt or a copper complex;
(b) a nitroxyl radical molecule-copper complex;
and combinations (a) and (b).

2. The oxidizing agent for 5-hydroxymethylcytosine according to claim 1, further comprising (c):
(c) one or more reaction promoters selected from the group consisting of pyridine, bipyridine, phenanthroline, ethylenediamine, propanediamine, imidazole, and derivatives thereof.

3. The oxidizing agent for 5-hydroxymethylcytosine according to claim 1, wherein the nitroxyl radical molecule is 2,2,6,6-tetramethylpiperidine-1-oxyl, 3-carbamoyl-2,2,5,5-tetramethyl-3-pyrrolin-1-yloxy, 2-azaadamantane-N-oxyl, 9-azabicyclo[3.3.1]nonane-N′-oxyl, or a derivative thereof.

4. A DNA demethylation analysis reagent, comprising the oxidizing agent for 5-hydroxymethylcytosine according to claim 1.

5. The DNA demethylation analysis reagent according to claim 4, further comprising a bisulfite.

6. A DNA demethylation analysis kit, comprising the DNA demethylation analysis reagent according to claim 5.

7. A method of oxidizing 5-hydroxymethylcytosine, comprising
mixing a test substance that may contain 5-hydroxymethylcytosine with the oxidizing agent for 5-hydroxymethylcytosine according to claim 1 in a reaction solution, and
incubating the reaction solution at a temperature of 4° C. to 90° C. for 1 to 100 hours to oxidize a hydroxy group in an allyl alcohol structure contained in 5-hydroxymethylcytosine.

8. A method of analyzing 5-hydroxymethylcytosine, comprising
mixing DNA with the oxidizing agent for 5-hydroxymethylcytosine according to claim 1 in a reaction solution,
incubating the reaction solution at a temperature of 4° C. to 90° C. for 1 to 100 hours to oxidize a hydroxy group in an allyl alcohol structure constituting 5-hydroxymethylcytosine, and
detecting 5-formylcytosine generated in the oxidation step.

9. The method according to claim 8, comprising cleaving a hydrogen bond contained in DNA, before performing the mixing step.

10. The method according to claim 9, comprising extracting DNA from a biological sample, before performing the hydrogen bond cleavage step.

11. The method according to claim 9, wherein in the hydrogen bond cleavage step, DNA is dissolved in a basic solution to cleave a hydrogen bond.

12. The method according to claim 8, wherein, in the detection step, 5-formylcytosine is detected by a bisulfite sequencing method.


SEQUENCE LISTING (4 sequences)

SEQ ID NO: 1 - DNA, 25 bp, Artificial
  primer
agtagtattttttaagttttggggg

SEQ ID NO: 2 - DNA, 36 bp, Artificial
  primer
caaatccctttataaaaaaaacaacaataacaacat

SEQ ID NO: 3 - DNA, 15 bp, Artificial
  synthetic
  misc_feature (8)..(8): n is 5-hydroxymethylcytosine
aaaaaagngaaaaaa

SEQ ID NO: 4 - DNA, 15 bp, Artificial
  synthetic
  misc_feature (8)..(8): n is 5-formylcytosine
aaaaaagngaaaaaa